(19) United States
(12) Patent Application Publication
(10) Pub. No.: US 2026/0364303 A1
(43) Pub. Date: Nov. 19, 2026

(54) METHOD AND SYSTEM FOR TREATING MENTAL CONDITION

(71) Applicant: YEDA RESEARCH AND DEVELOPMENT CO. LTD., Rehovot (IL)

(72) Inventors: Ehud AHISSAR, Rehovot (IL); Tchiya BEN JOSEPH, Rehovot (IL)

(21) Appl. No.: 19/676,176

(22) Filed: May 13, 2026

Related U.S. Application Data
(60) Provisional application No. 63/804,704, filed on May 13, 2025.

(51) Int. Cl. A61M 21/00; A61B 5/00; A61B 5/16

(52) U.S. Cl. CPC A61M 21/00; A61B 5/163; A61B 5/4836; A61B 5/7278; A61M 2021/005

(57) ABSTRACT

The present invention relates to therapeutic technologies, specifically to utilizing biofeedback and virtual reality technologies for developing personalized exposure therapy for treating mental health conditions such as Post-Traumatic Stress Disorder (PTSD). The claimed invention represents a system and method for treating mental conditions that improves the relevant technological field by providing and utilizing highly informative real-time biofeedback to reliably monitor therapy progress and dynamically adjust therapy session parameters. Specifically, the present invention provides a solution that monitors the reduction of avoidance behavior caused by anxiety and fear invoked by exposure to trauma-triggering situations, and further utilizes it as a feedback to alter therapy parameters. Such a solution allows users to influence the therapy session through their interaction with trauma-associated objects, potentially reducing feelings of helplessness associated with PTSD.

### CROSS REFERENCE TO RELATED APPLICATIONS

This application claims the benefit of priority under 35 U.S.C. § 119(e) of U.S. Provisional Patent Application No. 63/804,704, filed May 13, 2025, entitled “METHOD AND SYSTEM FOR TREATING MENTAL CONDITION”. The contents of the above application are all incorporated by reference as if fully set forth herein in its entirety.

### FIELD OF THE INVENTION

The present invention relates to therapeutic technologies, specifically to utilizing biofeedback and virtual reality technologies for developing personalized exposure therapy for treating mental health conditions such as Post-Traumatic Stress Disorder (PTSD).

### BACKGROUND OF THE INVENTION

Post-Traumatic Stress Disorder (PTSD) is a mental health condition triggered by experiencing or witnessing a traumatic event, such as a natural disaster, serious accident, terrorist act, war/combat, rape, or other violent personal assault. Individuals with PTSD may experience a range of symptoms, including intrusive memories, avoidance, negative changes in thinking and mood, and changes in physical and emotional reactions. The severity and duration of PTSD symptoms can vary, with some individuals recovering within a few months, while others may experience symptoms for much longer.

As known in the art, memories can be altered and influenced during the retrieval process, with sensory-motor conditions playing a significant role in the re-encoding of these memories. Traditional therapeutic methods aim to recreate environments similar to traumatic episodes to facilitate the transition from fragmented and painful memories to more coherent and less distressing ones.

Recent technological advancements have led to the development of new exposure therapies using virtual reality (VR), which help establish a sense of immersion and have shown positive results in reducing symptoms of PTSD.

However, these methods face significant challenges. Exposure therapy must be conducted safely, under close therapist supervision, and with the patient's continuous consent throughout the process. Known methods often have difficulty ensuring patient safety, frequently failing to detect distress during exposure to trauma-resembling conditions. Additionally, traumatic experiences are often accompanied by feelings of helplessness due to the passivity and lack of control over the situation. Typical VR-based therapeutic tools have limited active aspects of perception.

### SUMMARY OF THE INVENTION

Accordingly, there is a need for a system and method for treating mental conditions that would improve the relevant technological field by providing and utilizing highly informative real-time biofeedback to reliably monitor therapy progress and dynamically adjust therapy session parameters. Specifically, there is a need for the solution that would monitor the reduction of avoidance behavior caused by anxiety and fear invoked by exposure to trauma-triggering situations, and further utilize it as a feedback to alter therapy parameters. Such a solution would allow users to influence the therapy session through their interaction with trauma-associated objects, potentially reducing feelings of helplessness associated with PTSD. Furthermore, it would enable therapists to have better control over the progress of the therapy session. Consequently, this solution would significantly increase the safety and effectiveness of exposure therapy, leading to improved therapeutic outcomes for individuals with PTSD.

To address the aforementioned needs, the following is suggested.

In the general aspect, the present invention may be directed to a system for treating a mental condition. The system may include: an input means including at least one eye-tracking sensor configured to detect gaze points each representing a data sample indicating where a user is looking at a specific moment in time; an output means, configured to provide sensory stimuli to the user, said sensory stimuli being characterized by at least one parameter; at least one non-transitory memory device, wherein modules of instruction code are stored; and at least one processor associated with said at least one memory device, the input means and the output means, and configured to execute the modules of instruction code, whereupon execution of said modules of instruction code, the at least one processor may be configured to: based on the detected gaze points, continuously calculate a dwell time metric value; and dynamically alter a value of said at least one parameter, based on the calculated dwell time metric value.

In another general aspect, the present invention may be directed to a method for treating a mental condition. The method may include: detecting, using at least one eye-tracking sensor, a plurality of gaze points each representing a data sample indicating where a user is looking at a specific moment in time; based on the detected plurality of gaze points, continuously calculating a dwell time metric value; and providing, using output means, sensory stimuli to the user, said sensory stimuli being characterized by at least one parameter, while dynamically altering a value of said at least one parameter, based on the calculated dwell time metric value.

In some embodiments, the dwell time metric value may represent a duration of a dwell time period characterized by having the determined gaze points substantially within a specific area of interest (AOI).

In some embodiments, said AOI may be characterized by a fixed position in spatial coordinates within the field of view, so that said fixed position remains constant regardless of user’s head movement.

In some embodiments of the system, said at least one processor may be further configured to calculate the dwell time metric value by: defining a set of the gaze points determined withing a sliding time window of a predetermined duration; determining, within the defined set, one or more sequences of the gaze points characterized by positioning within the AOI; calculating duration of one or more dwell time sub-periods, each corresponding to a respective one of the determined sequences; and determining the duration of the dwell time period, based on the calculated duration of the one or more dwell time sub-periods.

Accordingly, in some embodiments of the method, said continuously calculating a dwell time metric value may include: defining a set of the gaze points determined withing a sliding time window of a predetermined duration; determining, within the defined set, one or more sequences of the gaze points characterized by positioning within the AOI; calculating duration of one or more dwell time sub-periods, each corresponding to a respective one of the determined sequences; and determining the duration of the dwell time period, based on the calculated duration of the one or more dwell time sub-periods.

In some embodiments, said duration of the dwell time period may be represented by at least one of (i) a total duration of all of said one or more dwell time sub-periods; (ii) an average duration of said one or more dwell time sub-periods; and (iii) a standard deviation of the duration of said one or more dwell time sub-periods.

In some embodiments of the system, said at least one processor may be further configured to dynamically alter the value of said at least one parameter correlatively to the determined duration of the dwell time period.

In some embodiments of the method, said dynamically altering the value of said at least one parameter may be performed correlatively to the determined duration of the dwell time period.

In some embodiments of the system, said at least one processor may be further configured to dynamically alter the value of said at least one parameter, provided that the determined duration of the dwell time period is below a predefined threshold value.

Accordingly, in some embodiments of the method, said dynamically altering the value of said at least one parameter is performed, provided that the determined duration of the dwell time period is below a predefined threshold value.

In some embodiments of the system, said at least one processor may be further configured to gradually adjust said predetermined duration of the sliding time window, correlatively to the determined duration of the dwell time period.

Accordingly, in some embodiments, the method may further include gradually adjusting said predetermined duration of the sliding time window, correlatively to the determined duration of the dwell time period.

In some embodiments, said sensory stimuli may include visual stimuli.

In some embodiments, said at least one parameter may be blurriness.

In some embodiments, said output means may include a Virtual-Reality (VR) headset configured to display a stereoscopic video content to the user and to track user’s head movement to adjust the stereoscopic video content based thereon.

Accordingly, in some embodiments, said visual stimuli may be provided as the stereoscopic video content.

In some embodiments, said AOI may be associated with a virtual object provided within the stereoscopic video content.

In some embodiments, said input means may include at least one motion controller configured to provide a motion input data element corresponding to the movement thereof, enabling interaction with the stereoscopic video content.

In some embodiments, said stereoscopic video content may include interactive tasks to be performed using said motion input data element, said interactive tasks requiring the user to retain visual focus withing the AOI in order to successfully complete said interactive tasks.

In some embodiments, said mental condition may be Post-Traumatic Stress Disorder (PTSD). Said AOI may be associated with a virtual object provided within the stereoscopic video content, said virtual object being associated with a traumatic experience of the user.

In some embodiments, said sensory stimuli may further include auditory stimuli.

In some embodiments, said at least one parameter may be loudness.

In some embodiments, said sensory stimuli may include haptic feedback.

In some embodiments, said at least one parameter may be an intensity of the haptic feedback.

In some embodiments, said input means further include at least one flow rate sensor configured to measure inhalation intensity of the user. Additionally, in such embodiments, said sensory stimuli may include olfactory stimuli. Accordingly, in some embodiments of the system, said at least one processor may be further configured to dynamically alter (i) a value of an intensity of the olfactory stimuli, based on the calculated dwell time metric value; and (ii) at least one of (a) an odor characterization; and (b) a hedonic tone, based on the measured inhalation intensity.

Accordingly, in some embodiments, the method may further include measuring, using at least one flow rate sensor, an inhalation intensity of the user, and dynamically altering (i) a value of an intensity of the olfactory stimuli, based on the calculated dwell time metric value; and (ii) at least one of (a) an odor characterization (also may be referred as odor characterization value); and (b) a hedonic tone, based on the measured inhalation intensity.

In some embodiments, the method may further include: administering an initial dose of a psychedelic compound to the user, monitoring, during a predetermined treatment efficiency evaluation period, the calculated dwell time metric value to determine an extent of changes therein; adjusting the initial dose based on the determined extent of changes; and administrating the adjusted dose of the psychedelic compound to the user.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic representation of a concept of the present invention, according to some embodiments;

FIG. 2 is a block diagram providing a general representation of a system for treating a mental condition, according to some embodiments;

FIG. 3 is a block diagram providing a detailed representation of the system for treating a mental condition, according to some embodiments; and

FIG. 4 is a flow diagram, depicting a method for treating a mental condition, according to some embodiments;

FIG. 5 is an example treatment protocol for combining psychedelic-assisted therapy with VR-based immersive exposure techniques, according to some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “defining”, “measuring”, “establishing”, “analyzing”, “checking”, “choosing”, “selecting”, “omitting”, “training”, “applying”, “forming” or the like, may refer to operation(s) and/or process(es) of a computer, a computing platform, a computing system, or other electronic computing device, that manipulates and/or transforms data represented as physical (e.g., electronic) quantities within the computer’s registers and/or memories into other data similarly represented as physical quantities within the computer’s registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

In relation to certain aspects of the invention, the present disclosure may refer to terms such as ‘environment’, ‘VR environment’, ‘video content’, ‘stereoscopic video content’, or similar. Within the context of this disclosure, these terms pertain to specific information provided to the user via sensory (visual) stimuli – for example, VR environment showing scenes and objects relevant to the traumatic experience of a specific user, or similar content of a video shown to the user etc. Therefore, in this context, these terms shall be considered equivalent.

Furthermore, the present disclosure may refer to terms such as “auditory stimuli”, “tactile stimuli”, “olfactory stimuli” etc., which, depending on the embodiments, may be provided separately to the “visual stimuli”, or may be integrated therewith – for example, VR environment combining visual scenes, objects, sounds, smells (odors) etc. relevant to the traumatic experience of a specific user.

Unless explicitly stated, the method embodiments described herein are not constrained to a particular order or sequence. Additionally, some of the described method embodiments or elements thereof can occur or be performed simultaneously, at the same point in time, concurrently, or iteratively and repeatedly.

The advent of advanced technologies, particularly in the field of biofeedback, presents a unique opportunity to develop more effective and engaging therapeutic interventions for treating PTSD. Biofeedback involves monitoring physiological signals and providing real-time feedback to the individual to help them learn to control and regulate these signals. When combined with various techniques of providing sensory stimuli to the user (e.g., with Virtual Reality (VR) technologies), biofeedback enables the creation of immersive and interactive therapeutic environments that are both engaging and effective.

The suggested invention leverages these advancements to provide a personalized therapeutic approach. It is suggested herein to adjust the sensory input in real-time based on each patient's motor output, in particular, based on specific metric of their eye movement pattern – a dwell time metric.

In the context of eye tracking, dwell time refers to the total amount of time a participant's gaze remains within a specific area of interest (AOI) on a visual stimulus. This metric is crucial for understanding visual attention and engagement with the content represented within the AOI.

Accordingly, it is suggested to use the dwell time metric as an informative biofeedback parameter to evaluate how the user reacts and interacts with an object (e.g., VR object) associated with their individual traumatic experience. Therefore, it is suggested that this metric may be highly representative of the user’s ability to cope with avoidance behavior, fear, and stress. It is further suggested to dynamically alter sensory stimuli parameters (e.g., for visual stimuli – blurriness, for auditory stimuli – loudness, etc.) in response to the dwell time metric, supporting the user in adapting to the trauma-triggering situation and thereby effectively reducing avoidance behavior.

As sensory stimuli parameters depend on the dwell time metric, their alteration may be performed so as to increase or decrease the immersion in the therapeutic environment, thereby giving the user and the therapist tools to control the therapy process. Therefore, the suggested approach helps to reduce feelings of helplessness associated with PTSD and ensures safety.

According to some embodiments of the present invention, the dwell time metric value may be represented by a duration of a dwell time period characterized by having the determined gaze points substantially within a specific area of interest (AOI). The gaze points may be detected using an eye tracking sensor and may indicate, in spatial coordinates, where a user is looking at a specific moment in time. Through an algorithm that continuously analyzes the gaze points and calculates dwell time metric (e.g., by defining a set of the gaze points determined withing a sliding time window of a predetermined duration, determining, within the defined set, one or more sequences of the gaze points characterized by positioning within the AOI; calculating duration of one or more dwell time sub-periods, each corresponding to a respective one of the determined sequences; and determining the duration of the dwell time period, based on the calculated duration of the one or more dwell time sub-periods), the suggested system causes the patient to experience changes in at least one parameter of the sensory stimuli, corresponding to the dwell time metric.

This method offers a more personalized and adaptive PTSD therapy regimen compared to conventional exposure therapy, which are less secure, controllable, adjustable and, thereby, less effective.

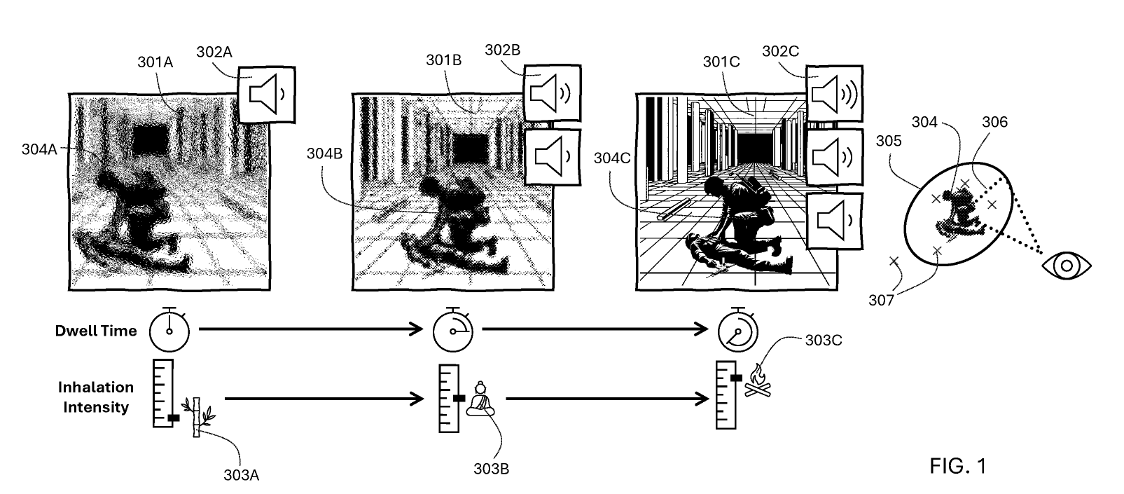

The concept of the present invention is further explained with reference to FIG. 1.

According to the concept, the system may provide various sensory stimuli to the user: e.g., visual stimuli 301A-301-C (e.g., a stereoscopic video content), auditory stimuli 302A-302C (e.g., spatial sound), and olfactory stimuli 303A-303C. Stimuli 301A-301C, 302A-302C and 303A-303C may be provided as a VR environment. The important aspect of the VR environment is to provide an object (represented as elements 304, 304A-304C) associated with the traumatic experience (trigger) of the patient (in the provided example – death of a soldier in a battlefield). It is further suggested to associate the object with an AOI (represented by element 305) within the VR environment, with respect to which the patient’s gaze behavior is evaluated.

Additionally, in some embodiments, stereoscopic video content (VR environment) may include interactive tasks to be performed using motion input data provided via motion controllers. For example, users may be requested to interact with the trauma-associated object (e.g., object represented by elements 304, 304A-304C), using motion controllers mimicking real-life hand motion and interaction (e.g., touching the object).

While using VR environment, user’s ocular motoric may be continuously monitored using at least one eye-tracking sensor (e.g., built into VR headset or a stand-alone one) configured to detect gaze points 307 each representing a data sample indicating where a user is looking at a specific moment in time.

Detected gaze points 307 may be further used to continuously calculate the dwell time metric. As indicated above, in some embodiments, the dwell time metric value may be represented by a duration of a dwell time period characterized by having the determined gaze points (e.g., gaze points 307) substantially within a specific AOI (e.g., AOI 305). In some embodiments, AOI 305 may be characterized by a fixed position in spatial coordinates within the field of view (e.g., 360-degree field of view), so that said fixed position remains constant regardless of user’s head movement. For example, AOI 305 could be an area around VR object 304 (also represented by elements 304A-304C) within the VR environment.

In some embodiments, the dwell time metric value may be calculated by: defining a set of the gaze points (such as gaze points 307) determined withing a sliding time window of a predetermined duration (may be defined depending of the strength of the PTSD symptoms, either manually by the therapist, or automatically by the system to ensure safe immersion into the VR environment); determining, within the defined set, one or more sequences of the gaze points characterized by positioning within the AOI (e.g., AOI 305); calculating duration of one or more dwell time sub-periods, each corresponding to a respective one of the determined sequences; and determining the duration of the dwell time period, based on the calculated duration of the one or more dwell time sub-periods.

It is known that ocular motor activity during visual concentration on a specific AOI is characterized by having a plurality of saccades – rapid, simultaneous movements of both eyes that shift the focus of gaze from one point to another, and plurality of ocular drifts – small, involuntary eye movement that occurs when the eyes are fixated on a single point. Depending on the embodiments, dwell time metric calculation may include total duration of sub-periods of time within the sliding time window and AOI (e.g., AOI 305), taking into account saccades, ocular drifts or both groups of ocular motor activity.

Depending on a specific embodiment, said duration of the dwell time period may be represented by, e.g., (i) a total duration of all of said one or more dwell time sub-periods; (ii) an average duration of said one or more dwell time sub-periods; and (iii) a standard deviation of the duration of said one or more dwell time sub-periods.

According to the concept of the present invention, the calculated dwell time metric may be further used as a biofeedback metric to dynamically alter a value of at least one parameter of the sensory stimuli. In particular, in some embodiments, at least one parameter of the sensory stimuli may be further dynamically altered correlatively to the determined duration of the dwell time period within AOI 305, or specific region thereof.

For example, AOI 305 may be predefined (or dynamically defined, as explained further below) by radius 306 from a predefined point considered a center of AOI 305 (e.g., associated with the geometrical center of object 304). Hence, it is suggested to calculate the distance of each determined gaze point 307 from the center of AOI 305, and, if the calculated distance is shorter than radius 306 or equal thereto, the respective point 307 may be considered positioned within AOI 305. Each gaze point 307 may be associated with a timestamp. Accordingly, the durations of sub-periods within the sliding time window may be determined as a difference between the timestamp of the latest of the determined gaze points 307 (determined withing the sliding time window) in the respective sequence and the earliest of the determined gaze points 307 in the respective sequence. Once point 307 is determined out of the AOI 305, it may indicate that the previous sub-period is ended, and its duration should end with the last point 307 determined within AOI 305. Accordingly, the calculation of the new sub-period may start only when the new point 307 positioned within AOI 305 is determined. All the time between the last point 307 within AOI 305 and the new point 307 within AOI 305 may thus not be taken into account when calculating the duration of the dwell time period. Consequently, the more points 307 that are determined within AOI 305 within the time window, the longer the duration of the dwell time period will be.

Accordingly, the calculated duration of the dwell time period may be indicative of how long the user may stay concentrated on the trauma-triggering object (e.g., object 304) within AOI 305 without switching visual attention to something else. Accordingly, the calculated duration of the dwell time period indicates how well the user copes with fear and avoidance behavior.

As further suggested herein, the parameters of the sensory stimuli may be dynamically altered correspondingly to the calculated dwell time metric (e.g., corresponding to the duration of the dwell time period). E.g., as shown in FIG. 1 (the left part), at the beginning of the therapeutic session, it may be hard for the patient to look directly at the trauma-associated object 304A. Accordingly, the majority of detected gaze points 307 will be outside of AOI 305 surrounding object 304A, and the calculated duration of the dwell time period will be low. To provide the user with the gradual immersion into the trauma-associated situation, it is further suggested that the parameters of the sensory stimuli are altered so as to have the highest blurriness level of visual stimuli (as shown by stereoscopic video content 301A) and the lowest loudness level of the auditory stimuli (audio content 302A) corresponding to the lowest value of the duration of the dwell time period.

In some embodiments, sensory stimuli may further include olfactory stimuli. It is further suggested to measure inhalation intensity of the patient, which also indicates the level of stress they experience while being immersed in the trauma-associated situation. Accordingly, it is further suggested to dynamically alter (i) a value of an intensity of the olfactory stimuli, based on the calculated dwell time metric value; and (ii) at least one of (a) an odor characterization (a parameter that refers to the specific description of what an odor of the olfactory stimuli smells like, such as “smoke”, “rose”, “cinnamon” etc.); and (b) a hedonic tone (a measure of the psychological impact an odor has on individuals, e.g., its perceived pleasantness or unpleasantness), based on the measured inhalation intensity. In some embodiments, it is further suggested to alter the odor characterization as an intensity of each of odors and/or a composition of several odors.

Accordingly, at the beginning of the therapeutic session, the inhalation intensity may be comparatively low and, as indicated above, the calculated duration of the dwell time period may also be low. Hence, according to some embodiments, it is suggested to alter parameters of the olfactory stimuli so that the lowest value of the duration of the dwell time period corresponds to the lowest intensity of the olfactory stimuli, and the lowest value of the inhalation intensity corresponds to the pleasant smell, having odor characterization that is not associated with the individual traumatic experience, as indicated by element 303A.

Then, as the therapeutic session proceeds, the patient becomes less stressed by the presence of the trauma trigger (e.g., object 304, 304A-304C), and the patient’s ocular-motor behavior may change. Specifically, as shown in FIG. 1 (the center part) the patient may become more focused on object 304B (accordingly, object 304B is shown in the center of the patient’s field of view, while object 304A is shown shifted to the side of the field of view), and may spend more time visually studying it. Accordingly, there may be more gaze points 307 detected within AOI 305 surrounding object 304B, and the calculated duration of the dwell time period will gradually increase. The parameters of the sensory stimuli may be altered correspondingly to the changes in the duration of the dwell time period: e.g., the blurriness level may gradually decrease, and the loudness level may gradually increase (as shown by stereoscopic video content 301B and audio content 302B). In some embodiments, the olfactory stimuli may also be altered correspondingly to the increase in the duration of the dwell time period. Specifically, the intensity of the olfactory stimuli may be gradually increased, correspondingly to the duration of the dwell time period. Accordingly, as the patient becomes more focused on the triggering situation, the measured inhalation intensity may gradually increase. Accordingly, as suggested herein, the odor characterization and/or hedonic tone parameters may be altered correspondingly, to provide neutral type of smells (as shown by element 303B).

Finally, as the therapeutic session progresses towards its conclusion, the patient becomes increasingly adapted to the traumatic trigger, resulting in changes to their ocular motor behavior. E.g., as shown in FIG. 1 (the right part), by the end of the session, the patient overcomes the psychological barrier and may observe the triggering object (e.g., object 304, 304C) more closely. Accordingly, the majority of the detected gaze points 307 may be within AOI 305 surrounding object 304C, and the calculated duration of the dwell time period will reach a certain maximum value. The parameters of the sensory stimuli may be altered correspondingly to the changes in the duration of the dwell time period: e.g., the blurriness level may reach its minimum (i.e., the stereoscopic video content 301C may be completely sharp), and the loudness level may reach its comfortable maximum (as shown by audio content 302C). In some embodiments, the olfactory stimuli may also be altered correspondingly to the increase in the duration of the dwell time period. Specifically, the intensity of the olfactory stimuli may be at its maximum level, corresponding to the maximum value of the duration of the dwell time period. Accordingly, as the patient studies the triggering situation more closely, the measured inhalation intensity may reach its maximum (corresponding to an average value for a stressed condition). Accordingly, as suggested herein, the odor characterization and/or hedonic tone parameters may be altered correspondingly, to provide an unpleasant type of smells, e.g., associated with the individual traumatic experience (e.g., in the provided example, odor characterization may be “blood”, “smoke” etc.) (as shown by element 303C). Finally, when the user resigns themselves to the traumatic situation, their inhalation intensity may return to normal values, and the olfactory stimuli parameters may change correspondingly.

Therefore, by dynamically altering values of the sensory stimuli parameters (such as blurriness level, loudness level, olfactory stimuli intensity, odor characterization, hedonic tone etc.) correlatively to the determined duration of the dwell time period, patients undergo gradual immersive experience. Furthermore, all the time the retain control over the therapeutic process – the immersive aspect thereof – as by simply stopping looking at the trauma-triggering object they may gradually reduce the effect of different stimuli (visual, auditory, olfactory) contributing to the feeling of immersion. And vice-versa – the more closely they start studying the trauma-triggering object, the stronger the effect of immersion becomes.

Therefore, by dynamically altering the values of sensory stimuli parameters (such as blurriness level, loudness level, olfactory stimuli intensity, odor characterization, hedonic tone etc.) in correlation with the determined duration of the dwell time period, patients undergo a gradual immersive experience. Furthermore, they retain control over the therapeutic process and its immersive aspects. By simply looking away from the trauma-triggering object, they can gradually reduce the effect of different sensory stimuli (visual, auditory, olfactory) contributing to the feeling of immersion. Conversely, the more closely they study the trauma-triggering object, the stronger the effect of immersion becomes, as all sensory stimuli become more intense.

On the other hand, therapists gain more control over the VR-based therapeutic session. They receive more data about patients' behavior (e.g., the dwell time metric) and can simultaneously observe how patients interact with trauma-triggering objects (e.g., object 304) on a separate screen. This allows therapists to intervene in the process by adjusting parameters such as the sliding time window duration, target area size, and enabling or disabling various stimuli separately.

As demonstrated, the dwell time metric may be used as informative real-time biofeedback to reliably monitor therapy progress and dynamically adjust therapy session parameters. Thereby, the solution may allow the users to influence the therapy session through their interaction with trauma-associated objects, potentially reducing feelings of helplessness associated with PTSD and would enable therapists to have better control over the progress of the therapy session. Consequently, this solution may significantly increase the safety and effectiveness of exposure therapy, leading to improved therapeutic outcomes for individuals with PTSD.

Additionally, in some embodiments, both therapists and patients may switch the trauma-associated VR content to something not associated with the trauma, if needed, or the system may be configured to do this automatically, e.g., when the duration of the dwell time period remains lower certain threshold value for a predefined period of time, signaling that the user is having troubles to proceed with the exposure therapy.

Additionally, in some embodiments, the suggested system may also comprise additional physiological sensors, such as heart rate monitors and skin conductance sensors, to provide more comprehensive biofeedback in the analysis of the patients’ stress level. The measurements received from such sensors may be used either of both for altering sensory stimuli parameters, or for interrupting the therapeutic session (the application of the sensory stimuli) if the stress level exceeds a predefined threshold.

It should be understood that when referring to alteration of the parameters of the sensory stimuli (e.g., blurriness and loudness levels) “correspondingly” to the duration of the dwell time period, depending on the embodiments, the values of said parameters may be altered proportionally, dynamically proportionally, exponentially, or according to another similar function, with respect to the calculated duration of the dwell time period.

It should be understood that although the system configuration using the above-described type of VR environment has demonstrated high efficiency, this specific type of VR environment is provided as a non-exclusive example only, and the present invention shall not be considered limited to this configuration. Furthermore, in some embodiments, sensory stimuli may not include visual stimuli. For example, the user may look at real objects and perform various interactive tasks using them, while receiving ocular biofeedback through other types of sensory stimuli, such as auditory or haptic feedback.

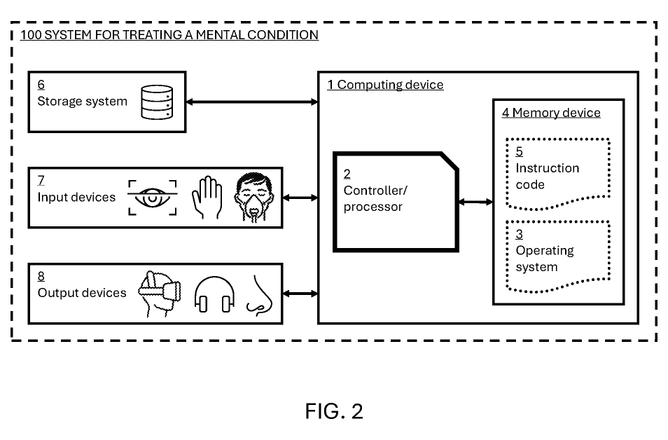

Reference is now made to FIG. 2, which is a block diagram providing a general representation of system 100 for treating a mental condition (e.g., PTSD), according to some embodiments.

In some embodiments, system 100 may include computing device 1.

Computing device 1 may include a processor or controller 2 that may be, for example, a central processing unit (CPU) processor, a chip or any suitable computing or computational device, an operating system 3, a memory device 4, instruction code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, system 100 according to embodiments of the invention.

Operating system 3 may be or may include any code segment (e.g., one similar to instruction code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It is noted that an operating system 3 may be an optional component, e.g., in some embodiments, a system may include a computing device that does not require or include an operating system 3.

Memory device 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory device 4 may be or may include a plurality of possibly different memory units. Memory device 4 may be a computer or processor non-transitory readable medium, or a computer non-transitory storage medium, e.g., a RAM. In one embodiment, a non-transitory storage medium such as memory device 4, a hard disk drive, another storage device, etc. may store instructions or code which when executed by a processor may cause the processor to carry out methods as described herein.

Instruction code 5 may be any executable code, e.g., an application, a program, a process, task, or script. Instruction code 5 may be executed by processor or controller 2 possibly under control of operating system 3. For example, instruction code 5 may be a standalone application or an API module that may be configured to: detect, using at least one eye-tracking sensor, a plurality of gaze points each representing a data sample indicating where a user is looking at a specific moment in time; based on the detected plurality of gaze points, continuously calculate a dwell time metric value; and provide, using output means, sensory stimuli to the user, said sensory stimuli being characterized by at least one parameter, while dynamically altering a value of said at least one parameter, based on the calculated dwell time metric value, as described herein. Although, for the sake of clarity, a single item of instruction code 5 is shown in FIG. 2, a system according to some embodiments of the invention may include a plurality of executable code segments or modules similar to instruction code 5 that may be loaded into memory device 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Various types of input and output data may be stored in storage system 6 and may be loaded from storage system 6 into memory device 4 where it may be processed by processor or controller 2. In some embodiments, some of the components shown in FIG. 2 may be omitted. For example, memory device 4 may be a non-volatile memory having the storage capacity of storage system 6. Accordingly, although shown as a separate component, storage system 6 may be embedded or included in memory device 4.

Input devices 7 may be or may include any suitable input devices, components, or systems, e.g., eye-tracking sensors, motion controllers, inhalation flow meters etc. In some embodiments, input devices 7 may further include additional physiological sensors, such as heart rate monitors and skin conductance sensors, to provide more comprehensive biofeedback and to further alter at least one parameter of sensory stimuli based on signals therefrom. Input devices 7 may also include, e.g., a detachable keyboard or keypad, a mouse and the like. Output devices 8 may include one or more (possibly detachable) displays or monitors, headphones, speakers, olfactory (scent) devices (olfactometers) and/or any other suitable output devices. Various input and output devices (7 and 8) may be integrated into a single device, such as a Virtual-Reality (VR) headset. This headset may be configured to display stereoscopic video content and play sounds (acting as output device 8), while also tracking the user's head movements (acting as input device 7) to adjust the video content based thereon. Any applicable input/output (I/O) devices may be connected to computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

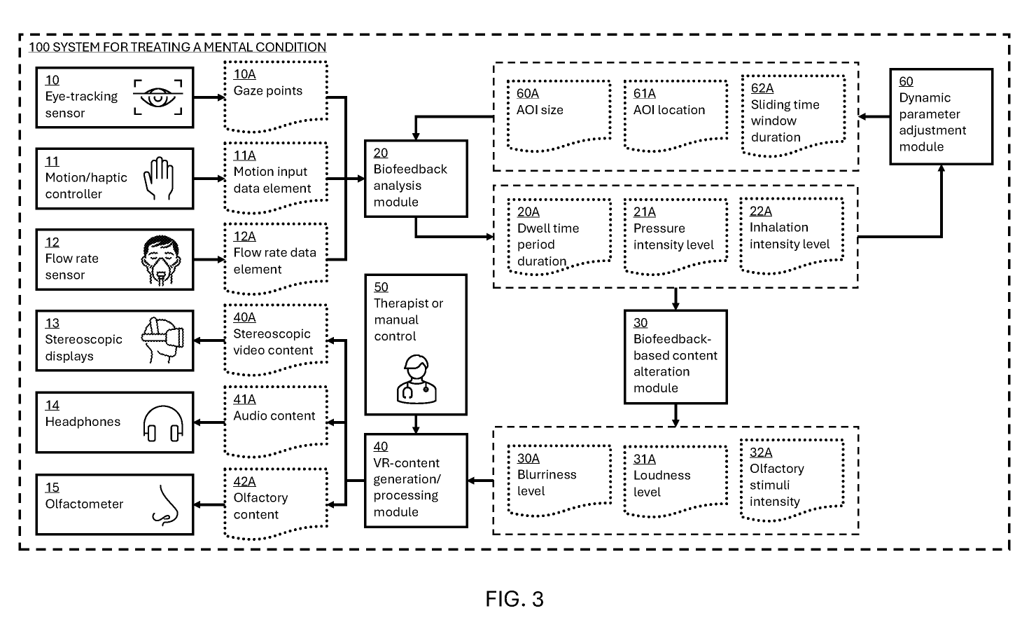

Reference is now made to FIG. 3, which is a block diagram providing a detailed representation of system 100 for treating a mental condition, according to some embodiments.

According to some embodiments of the invention, system 100 may be implemented as a combination of software and hardware modules. For example, system 100 may be or may include computing devices such as element 1 of FIG. 2. Furthermore, system 100 may be adapted to execute one or more modules of instruction code (e.g., element 5 of FIG. 2) to request, receive, analyze, calculate and produce various data.

As further described in detail herein, system 100 may be adapted to execute one or more modules of instruction code (e.g., element 5 of FIG. 2) in order to perform steps of the claimed method.

As shown in FIG. 3, arrows may represent the flow of one or more data elements to and from system 100 and/or among modules or elements of system 100. Some arrows have been omitted in FIG. 3 for the purpose of clarity.

As shown in FIG. 3, in some embodiments, system 100 may include eye-tracking sensor 10. Eye-tracking sensor 10 may be configured to detect gaze points 10A (such as gaze points 307, discussed with reference to FIG. 1) each representing a data sample indicating where a user is looking at a specific moment in time.

Eye-tracking sensor 10 may, e.g., include infrared camera that capture reflections from the user's eyes or electrooculography (EOG) sensor that measure the electrical activity of the eye muscles. Sensor 10 may be embedded within the VR headset or attached externally, and may communicate wirelessly with computing device 1 (as shown in FIG. 2) to ensure seamless integration.

It should be understood that the present invention is not limited to a specific eye-tracking sensor (sensor 10). Any known eye-tracking sensor suitable for the purposes of this invention (e.g., configured to detect gaze points) may be used herein. A person skilled in the art will recognize which known eye-tracking sensors are applicable and how to implement them.

In some embodiments, system 100 may further include motion controller 11.

Motion controller 11 may be utilized with a Virtual Reality (VR) headset to enhance user interaction and immersion. Various types of motion controllers may be used herein. These controllers may include handheld devices equipped with sensors to detect position and orientation, allowing for precise tracking of hand movements. Additionally, motion controllers can incorporate haptic feedback mechanisms to provide tactile sensations, that may be used as sensory stimuli for the therapeutic approach suggested herein, further enriching the VR experience. Other types of motion controllers may include wearable devices, such as gloves or suits, embedded with sensors to capture full-body movements. These controllers can communicate wirelessly with the VR headset, ensuring seamless integration and real-time response to user actions. The versatility of these motion controllers allows for a wide range of therapeutic applications. E.g., the user may ‘touch’ the VR object associated with trauma (e.g., object 304, as discussed with reference to FIG. 1) and receive a haptic feedback (the intensity of which may be adjusted based on the dwell time metric, as explained herein), which can make the VR experience more immersive.

In some embodiments, system 100 may further include flow rate sensor 12.

Flow rate sensor 12 may be any type of commonly known device used to measure the rate at which a fluid or gas flows through a system. In the context of the present invention, such sensors may be considered crucial for monitoring and adjusting the intensity of olfactory stimuli based on the intensity of the user's inhalation. In some embodiments, flow rate sensor 12 may be or may include differential pressure sensor, which measure the pressure difference across a constriction to determine flow rate, or thermal mass flow sensor, which measure the heat carried away by the flowing gas to calculate its velocity. These are common types of flow rate sensors used in VR applications. They provide accurate and real-time data, enhancing the immersive experience by dynamically adjusting the sensory stimuli to match the user's breathing patterns.

In some embodiments, system 100 may include stereoscopic displays 13.

Stereoscopic displays 13 can be integrated with the Virtual Reality (VR) headset to provide a more immersive and realistic visual experience. As known, these displays work by presenting two slightly different images to each eye, mimicking the way human binocular vision perceives depth. This technique creates a three-dimensional effect, allowing users to perceive depth and spatial relationships in the virtual environment. Stereoscopic displays typically use dual lenses or screens to achieve this effect, ensuring that each eye receives the appropriate image. The use of stereoscopic technology enhances the sense of presence in VR environment, making it ideal for exposure therapy purposes. By accurately replicating the way human see the real world, stereoscopic displays significantly improve the realism and engagement of VR content, which, when combined with the suggested Oculomotor Biofeedback (OBF), may provide further improvement to the relevant technological field. This improvement is achieved by increasing the efficiency of system 100, accelerating the therapeutic effect, and providing sustained fixation of the therapy results, as, by using such immersive VR technology, therapy is conducted through the interconnection between motor control and visual focus, mirroring real-life scenarios.

In some embodiments, system 100 may further include headphones 14.

Various types of headphones 14 may be used in system 100. In some embodiments, headphones 14 can be integrated with the VR headset to enhance the auditory experience and provide immersive soundscapes. These headphones may include over-ear, on-ear, or in-ear designs, each offering different levels of sound isolation and comfort. Some headphones may feature spatial audio technology, which simulates 3D sound by adjusting the audio output based on the user's head movements. This technology enhances the sense of presence and realism in VR therapy applications. Furthermore, noise-canceling features can be incorporated to minimize external distractions, allowing users to fully immerse themselves in the virtual world.

In some embodiments, system 100 may further include olfactometer 15.

Olfactory devices, also known as olfactometers, are used to deliver controlled scents in virtual reality (VR) environments, enhancing the immersive experience by stimulating the sense of smell. Olfactometer 15 may be or may include any common type of olfactometers used in VR application, such as valve-based olfactometers, which mix and release different odors through a series of valves, and miniaturized odor generators, which can be integrated into VR headsets. These devices often use technologies such as heating perfumed paraffin wax or releasing scent cartridges to produce a wide range of odors. By dynamically adjusting the intensity and type of scents, olfactometer 15 may significantly contribute to creating a more realistic and engaging VR experience, further contributing to the abovementioned improvement of the relevant technological field.

Accordingly, in some embodiments, system 100 may include a VR headset (comprising stereoscopic displays 13 and headphones 14 and having motion controller 11, eye-tracking sensor 10, flow-rate sensor 12 and olfactometer 15 connected thereto).

In some embodiments, system 100 may include biofeedback analysis module 20. In some embodiments, biofeedback analysis module 20 may be configured to continuously receive gaze points 10A from eye-tracking sensor 10. Biofeedback analysis module 20 may be further configured to receive preset parameters, defining an AOI in spatial coordinates, with respect to which the duration of the dwell time period may be calculated (e.g., such as AOI 305, discussed with reference to FIG. 1). These preset parameters may, e.g., include AOI size 60A and AOI location 61A. In some nonlimiting embodiments, AOI may be characterized by a fixed position in spatial coordinates within the field of view, so that said fixed position remains constant regardless of user’s head movement, as discussed with reference to FIG. 1.

In some embodiments, said preset parameters may further include sliding time window duration 62A, defining the period of observation for further gaze point dispersion evaluation.

Biofeedback analysis module 20 may be further configured to continuously define set of the gaze points 10A that were determined within the sliding time window of the preset duration 62A. Module 20 may be further configured to determine the dwell time metric value represented by duration 20A of a dwell time period characterized by having determined gaze points 10A substantially within a specific AOI (e.g., defined by size 60A and location 61A, such as AOI 305 discussed with reference to FIG. 1).

In particular, in some embodiments, module 20 may be further configured to calculate the dwell time metric value by: defining a set of gaze points 10A determined withing a sliding time window of predetermined duration 62A; determine, within the defined set, one or more sequences of gaze points 10A characterized by positioning within AOI (e.g., defined by size 60A and location 61A, such as AOI 305 discussed with reference to FIG. 1); calculating duration of one or more dwell time sub-periods, each corresponding to a respective one of the determined sequences; and determining duration 20A of the dwell time period, based on the calculated duration of the one or more dwell time sub-periods. E.g., in some embodiments, module 20 may be further configured to calculate duration 20A of the dwell time period as at least one of (i) a total duration of all of said one or more dwell time sub-periods; (ii) an average duration of said one or more dwell time sub-periods; and (iii) a standard deviation of the duration of said one or more dwell time sub-periods. In some embodiments, to determine positioning of gaze points 10A within or outside AOI 305, module 20 may calculate distance from each point to the center of AOI 305, based on the special coordinates thereof, and evaluate whether the determined distance is longer or shorter than the radius of AOI 305 (as determined by size 60A).

It should be understood that the calculation of the “duration of the dwell time period” is not limited to any specific function of the timestamps of gaze points 10A and the calculated distances to the center of the AOI. E.g., in some embodiments, it may include various mathematical transformations of timepoints, durations of sub-periods, number of sub-periods within the time period, number and duration of ocular drifts, saccades etc. (e.g., normalization of some of these parameters). Furthermore, depending on the embodiments, it may be represented both in absolute and relative units of measurements. E.g., in some embodiments, the duration of the dwell time period may be expressed as a percentage, e.g., a percentage corresponding to duration 62A of the sliding time window.

In some embodiments, as additional parameter, module 20 may further output pressure intensity level 21A. The present invention shall not be considered limited with respect to any particular way of calculating pressure intensity level 21A. E.g., module 20 may be configured to calculate, based on motion input data elements 11A, how hard the user squeezes their hands (or use different hand movement pattern), which may also depend on the stress level. E.g., in some embodiments, module 20 may be configured to calculate pressure intensity level 21A as a normalized value of the average pressure values provided by motion data elements 11A detected within the sub-periods. Said normalized value may be calculated by module 20 with respect to the certain predefined threshold.

In some embodiments, module 20 may be further configured to output inhalation intensity level 22A. The present invention shall not be considered limited with respect to any particular way of calculating inhalation intensity level 22A. E.g., module 20 may be configured to calculate, based on flow rate data elements 12A, how intense the user inhales (e.g., inhale/exhale frequency, inhalation volume etc.), which may also depend on the stress level. E.g., in some embodiments, module 20 may be configured to calculate inhalation intensity level 22A as a normalized value of the average flow rate values provided by flow rate data elements 12A detected within the sub-periods. Said normalized value may be calculated by module 20 with respect to the certain predefined threshold.

In some embodiments, system 100 may further include biofeedback-based content alteration module 30. Biofeedback-based content alteration module 30 may be configured to receive calculated duration 20A of the dwell time period. Module 30 may be further configured to dynamically alter a value of at least one parameter of the sensory stimuli provided to the user, e.g., via stereoscopic displays 13, headphones 14 and olfactometer 15, based on the calculated dwell time metric (e.g., duration 20A of the dwell time period). E.g., module 30 may be further configured to alter the values of said parameters correlatively to the duration 20A of the dwell time period. For example, module 30 may store a specific predefined function from the duration 20A of the dwell time period to the value of the desired parameter. E.g., module 30 may alter the desired parameter proportionally, dynamically proportionally, exponentially etc. to the duration 20A. In some embodiments, said parameter may include blurriness level 30A (for the visual stimuli provided via stereoscopic displays 13) and/or loudness level 31A (for the auditory stimuli provided via headphones 14), and intensity 32A of the olfactory stimuli (for the olfactory stimuli provided via olfactometer 15).

In some embodiments, said sensory stimuli may also include tactile stimuli (haptic feedback provided by motion controller 11), and the parameters of the sensory stimuli may accordingly include intensity of the haptic feedback (not shown).

In some embodiments, the parameters of the auditory stimuli may also include the Head-Related Transfer Function (HRTF) adjustment. HRTF is the parameter of spatial audio that can be adjusted to control the extent to which the audio panorama changes in response to the user's head movements. By adjusting the HRTF, system 100 can either maintain a stable audio environment or create a dynamic soundscape that shifts dramatically with head motion away and to the target area. This capability may decrease or increase the immersive effect of user's visual concentration on the AOI radiating sound within the 3D environment, thereby improving the overall immersive experience and effectiveness of system 100 for treating the mental condition.

Module 30 may be configured to perform the alteration of the parameters of the sensory stimuli, e.g., as discussed with reference to FIG. 1.

Additionally, in some embodiments, module 30 may be further configured to receive a predefined threshold value, defining situations where duration 20A is within the acceptable region, meaning that the user experiences no problem keeping the visual attention within the AOI and therefore no alteration of the at least one parameter of the sensory stimuli is necessary; and situations where duration 20A is within the sub-normal region, meaning that the user still experiences difficulties keeping the visual attention on the AOI and, hance, said alteration is necessary. Accordingly, in some embodiments, module 30 may be further configured to dynamically alter the value of said at least one parameter, provided that the determined duration 20A is below the predefined threshold value.

In some embodiments, system 100 may further include VR content generation/processing module 40.

VR content generation/processing module 40 may be configured to generate stereoscopic video content 40A, audio content 41A, and olfactory content 42A (e.g., may include program instructions for olfactometer 15 on how to adjust the olfactory stimuli) to provide an immersive VR therapeutic environment to the user (same as explained with reference to FIG. 1), e.g., using stereoscopic displays 12, headphones 13, and olfactometer 15, respectively, which may be, for example, integrated in the VR headset. Accordingly, the VR headset may be configured to display stereoscopic video content 40A to the user and to track user’s head movement to adjust stereoscopic video content 40A based thereon, as well as providing auditory stimuli (audio content 41A) and olfactory stimuli (olfactory content 42A).

Additionally, in some embodiments, motion controller 11 may be configured to provide, to VR content generation/processing module 40, motion input data elements 11A corresponding to the movement of the motion controller 11 (e.g., coordinates of movement, signals from gyroscope sensors integrated in the controller 11 etc.), enabling interaction with stereoscopic video content 40A.

In some embodiments, module 50 may be further configured to generate stereoscopic video content 50A including interactive tasks to be performed using motion controller 11, i.e., based on motion input data elements 11A. Such interactive tasks may require the user to retain visual focus within the AOI (as explained with reference to FIG. 1), e.g., to ‘touch’ the VR object (e.g. object 304, described with reference to FIG. 1), while looking at it.

In some embodiments, system 100 may further include dynamic parameter adjustment module 60. Module 60 may be configured to receive duration 20A of the dwell time period. Using duration 20A, module 60 may evaluate how long a specific user may retain visual attention within the AOI. In some embodiments, if the user continuously keeps their gaze within the AOI, the duration 20A becomes longer and it indicates that the user proceeds with the therapy successfully. In such situations, to further assist users in their progress, module 60 can gradually adjust at least one of the following parameters correlatively to duration 20A of the dwell time period: (i) predetermined size 60A of the AOI; and (ii) predetermined duration 62A of the sliding time window. For example, the size 60A of the AOI may be decreased, and the duration 62A of the sliding time window may be increased. The same may apply to opposite scenarios. When duration 20A is too low, it may indicate that the current settings for size 60A and duration 62A are too difficult for the user. In such cases, module 60 may be configured to gradually increase size 60A and decrease duration 62A. Module 60 may continue these adjustments until the desired condition for effective therapy is achieved (e.g., until duration 20A will be at least 20% of duration 62A).

Such dynamic adjustment may further contribute to the abovementioned improvement of the technological field, by further improving system 100 efficiency. It creates a more personalized exposure therapy approach, accelerating the therapy process for individuals with varying levels of PTSD symptoms and ensuring sustained fixation of the results of the therapy for each case.

In some embodiments, system 100 may further include, or configured to receive input from additional physiological sensors (not shown), such as heart rate monitors and skin conductance sensors, to provide more comprehensive biofeedback and to further alter at least one parameter of sensory stimuli based on signals therefrom.

In some embodiments, system 100 may include therapist or manual control module 50. Module 50 may be configured to output information to a stand-alone display, such as the same video content 50A that the user is currently seeing (including any alterations to the visual stimuli parameters). It may also output information about the current dwell time metric value, the user's progress (e.g., how quickly the user improves duration 20A of the dwell time period), and additional data from various physiological sensors (e.g., heart rate monitors and skin conductance sensors). This output information can be reviewed by a therapist. Module 50 can also receive manual adjustments to the AOI size 60A, AOI location 61A, sliding time window duration 62A etc., allowing the therapist to intervene in the therapy if necessary.

To ensure safety, module 50 may be further configured to change the VR content (e.g., video content 40A, audio content 41A, and olfactory content 42A) from trauma-associated one to completely irrelevant, positive one. This function may be applied either or both by the user and therapist, when it is necessary for the user to rest from the therapy. Additionally or alternatively, in some embodiments, module 50 may be configured to switch to the ‘positive’ content, based on the measurements from sensors monitoring parameters indicative of the stress level of the patient, like heart-rate monitor, and flow rate sensor, to ensure safety, when patients condition becomes critical.

In some embodiments, module 50 may be further configured to generate and alter the VR environment individually for the traumatic experience of each patient, in order to further improve the efficiency thereof. To perform that, module 50 may further include machine-learning (ML)-based module, e.g., based on Large Language Models (LLM) and Generative models (like ChatGPT or Copilot).

As known, generative models are designed to generate new content based on the input they receive. They can create text, images, and other types of data, e.g., stereoscopic video content 40A.

Large language model are a type of generative model specifically trained on vast amounts of text data to understand and generate human-like language. They use deep learning techniques to predict and generate content that is coherent and contextually relevant to the input.

In some embodiments, verbal session with the specific patient may be conducted before applying system 100. In such section, the therapist may ask various questions about the traumatic experience of a particular user. Such session may be recorded, and the transcription thereof may be created. Then, this transcription may be used as an input for the ML-based model of module 50, to generate VR environment corresponding to the traumatic scenes discussed during the session. Accordingly, module 50 may generate such environment and then apply it, as discussed above.

In such embodiments, the suggested invention may further contribute to the improvement of the relevant technological field by creating personalized immersive experience, thereby increasing efficiency of system 100 in treating a mental condition, such as PTSD.

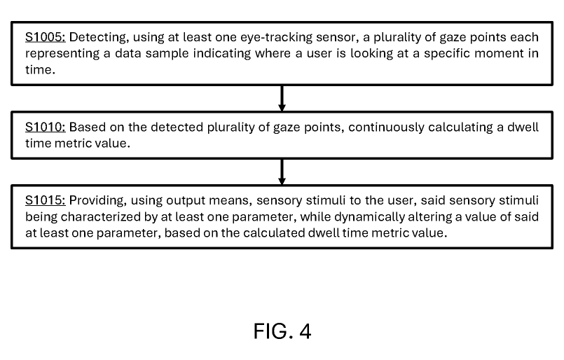

Referring now to FIG. 4, a flow diagram is presented, depicting a method for treating a mental condition, by at least one processor (e.g., processor 2 of FIG. 2), according to some embodiments.

As shown in step S1005, the at least one processor (e.g., such as processor 2 of FIG. 2) may detecting, using at least one eye-tracking sensor (e.g., eye-tracking sensor 10, as discussed with reference to FIG. 3), a plurality of gaze points (e.g., gaze points 307 and 10A, as discussed with reference to FIGS. 1 and 3, respectively) each representing a data sample indicating where a user is looking at a specific moment in time. Step S1005 may be carried out by eye-tracking sensor 10 (as described with reference to FIG. 3).

As shown in step S1010, the at least one processor (e.g., such as processor 2 of FIG. 2) may continuously calculate, based on the detected plurality of gaze points (e.g., gaze points 307 and 10A, as discussed with reference to FIGS. 1 and 3, respectively), a dwell time metric value (e.g., duration 20A of the dwell time period, as discussed with reference to FIGS. 1 and 3). Step S1010 may be carried out by biofeedback analysis module 20 (as described with reference to FIG. 3).

As shown in step S1015, the at least one processor (e.g., such as processor 2 of FIG. 2) may provide, using output means (e.g., stereoscopic displays 13, headphones 14, and olfactometer 15, as discussed with reference to FIG. 3), sensory stimuli (e.g., stereoscopic video content 40A, audio content 41A and olfactory content 42A, as discussed with reference to FIG. 3) to the user, said sensory stimuli being characterized by at least one parameter (e.g., blurriness level 30A, loudness level 31A, olfactory stimuli intensity 32A, as discussed with reference to FIG. 3), while dynamically altering a value of said at least one parameter, based on the calculated dwell time metric value (e.g., duration 20A of the dwell time period as discussed with reference to FIGS. 1 and 3). Step S1015 may be carried out by biofeedback-based content alteration module 30 and VR content generation/processing module 40 (as described with reference to FIG. 3).

Recent research has shown that psychedelic compounds like MDMA and psilocybin can significantly enhance the effectiveness of therapeutic sessions for treating PTSD. When used in controlled settings, these compounds facilitate emotional openness, reduce fear, and promote introspection, which can be particularly beneficial during therapy. MDMA has been found to reduce fear and increase emotional engagement, making it easier for patients to process traumatic memories. Clinical trials have shown that MDMA-assisted therapy can lead to significant reductions in PTSD symptoms. Psilocybin induces altered states of consciousness that can help patients gain new perspectives on their trauma. Studies have demonstrated its efficacy in reducing anxiety and depression, which are often collateral to PTSD. The known therapeutic process typically involves preparation sessions, the psychedelic dosing session, and integration sessions. Preparation sessions involve meeting with therapists to set intentions, explore expectations, and develop strategies for navigating the psychedelic experience. During the dosing session, the psychedelic compound is controllably administered, allowing patients to fully engage with their inner experiences. Integration sessions follow the dosing session and focus on consolidating the insights gained during the psychedelic experience and applying them to daily life.

Psychedelics help patients access and process deep-seated emotions and memories, which can be difficult to reach through traditional therapy alone. These compounds promote neuroplasticity, allowing the brain to form new connections and pathways, which can facilitate healing and recovery from trauma.

Although psychedelic psychotherapy has demonstrated efficacy, the application of psychedelic compounds necessitates precise dosing selection and a controlled therapeutic environment, which continues to be a subject of research.

In some embodiments of the present invention, it is suggested to combine the application of psychedelic compounds with the approach of providing sensory stimuli to the user and adjusting them based on ocular biofeedback, as discussed above. This combination may offer a highly effective method for treating various mental conditions, e.g., PTSD.

In particular, in some embodiments, the suggested method of treating a mental condition may further include: administering an initial dose of a psychedelic compound (such as MDMA, ibogaine, psilocybin, and cannabis) to the user. Depending on the embodiment, the administration may occur before the therapeutic session (which includes exposure therapy using a VR-based immersive environment, as described above), during the session (e.g., after the initial short VR-based immersive sub-session and before the subsequent VR-based immersive sub-session), and/or between the subsequent sessions. The method may further include monitoring, during a predetermined treatment efficiency evaluation period (e.g., corresponding to the length of the session or a sub-session, e.g., 10-30 minutes), the calculated dwell time metric value (e.g., duration 20A of a dwell time period, as described with reference to FIG. 3) to determine an extent of changes therein (e.g., whether the user improved the dwell time metric (the values increased), and how significant is the improvement). E.g., the extent of changes may be calculated as (i) a percentage change between the initial values (e.g., averaged initial values) and the final values (e.g., averaged final values); (ii) a standard deviation of the dwell time metric values over the treatment efficiency evaluation period; (iii) a coefficient of variation (CV) (expresses the standard deviation as a percentage of the mean) etc.

The method may further include adjusting the initial dose of the psychedelic compound based on the determined extent of changes in the calculated dwell time metric value. To do that, certain reference values of the extent of changes may be used.

For example, the reference values of the extent of changes that the user may demonstrate during the treatment efficiency evaluation period can be determined during a preliminary session. Such a preliminary session may include the VR-based immersive therapy (as described above), without administering the psychedelic compound. Accordingly, the extent of changes in the dwell time metric may be calculated during the treatment efficiency evaluation period of the preliminary session, to evaluate the user’s treatment progress before applying any psychedelic compounds. Then, in the following session, the same evaluation may be performed but after the administration of the psychedelic compounds. Next, the extent of changes of the preliminary session may be used as a reference, to be compared with the extent of changes of the following session, thereby evaluating how the administration of the psychedelic substances affected the treatment progress.

If the session that includes psychedelic substance administration demonstrates significantly higher extent of changes in the calculated dwell time metric (obviously, the extent of changes itself must be positive: the duration 20A of the dwell time period must increase during each session) compared to the reference values, it may be decided to continue with the initial dose. If the session that includes psychedelic substance administration demonstrates zero or insignificant increase in the extent of changes in the calculated dwell time metric compared to the reference values, it may be decided to increase the initial dose. Finally, if the session that includes psychedelic substance administration demonstrates a decrease in the extent of changes in the calculated dwell time metric compared to the reference values, it may be decided to decrease the initial dose. This adjustment may be necessary if the dose is too large for a specific user or if the user is particularly susceptible to the psychedelic substance. It shall be understood that the adjustment of the initial dose may be determined continuously, proportionally to the increase and decrease of the extent of changes.

Additionally or alternatively, the reference values may be determined based on the average or common extent of changes in the dwell time metric exhibited by users with “medium” severity of PTSD symptoms (e.g., determined from statistically accumulated data). These reference values may further be adjusted based on the user’s age, gender, type of psychological trauma, etc.

The method may further include administering the adjusted dose of the psychedelic compound to the user. As indicated above, depending on the embodiment, this may be done before the following session or within a single session (between two subsequent sub-sessions).

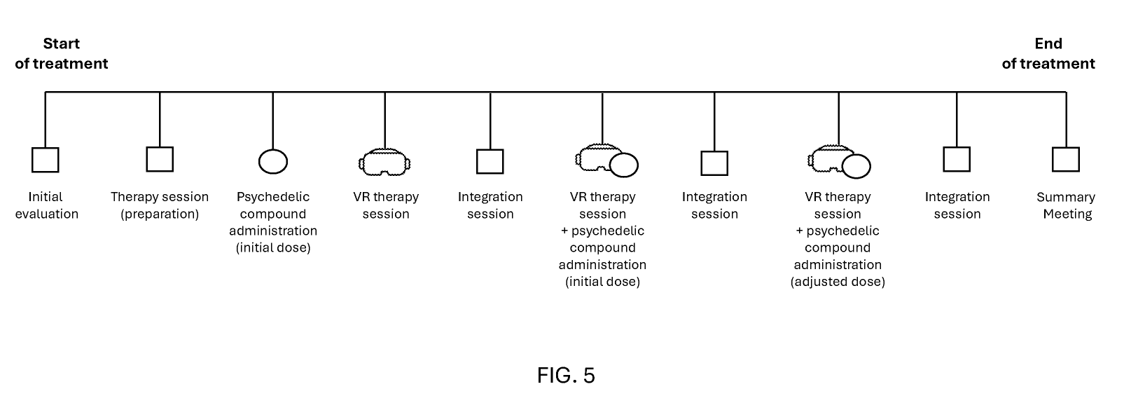

Reference is now made to FIG. 5, showing example treatment protocol for combining psychedelic-assisted therapy with VR-based immersive exposure techniques, according to some embodiments of the present invention.

As demonstrated in FIG. 5, the treatment protocol may start with the initial evaluation session, including an initial meeting. Then, the protocol may proceed with a preparation session to set intentions, explore expectations, and develop strategies for navigating the psychedelic-assisted VR experience. Next, the protocol may include a separate session for administering the psychedelic compound (at the initial dose) to assess the patient's tolerance and another separate session to familiarize them with the VR-based therapy approach. Then, the protocol may include two sessions that include the administration of the psychedelic compound followed the VR-based therapy, wherein, in the first session, the compound is administered in the initial dose, while in the second session, the compound is administered in the adjusted dose (the dose may be adjusted based on the extent of changes in the dwell time metric monitored during the first session, as explained above). Each session that includes VR-based therapy may be followed by an integration session. The goal of this session is to consolidate the insights gained through the VR-based immersive experience (with or without psychedelic compound administration) and to help the patient apply these changes to their daily life.

The protocol may end with a summary meeting, further consolidating the results of the entire treatment plan.

As can be seen, the embodiments of the present invention that include administering psychedelic substances may provide a synergic effect, significantly improving the respective technological field. On the one hand, the application of psychedelic substances improves the efficiency of the suggested VR-based immersive exposure therapy, making patients more open and susceptible to overcoming trauma-associated problems. On the other hand, the suggested VR-based immersive exposure therapy, in particular, the usage of the informative OBF metric as suggested herein (the dwell time metric) provides for a safe and reliably controlled treatment environment, where the monitored metric may be used as feedback for dose adjustment. Thereby the suggested solution enables a personalized treatment plan, which is necessitated by the fact that both the severity of the psychological trauma and the susceptibility to the psychedelic compounds significantly vary between patients. The suggested combination of techniques enables precise selection of the effective dosage based on the VR-based therapy progress observed through sessions.

It should be understood that although the efficiency of the present invention is discussed in relation to treating patients with PTSD, its scope is not limited to this specific application, and, in some embodiments, the present invention may be applied for treating other mental conditions, e.g., phobias.

As can be seen from the provided description, the claimed invention represents a system and method for treating mental conditions that improves the relevant technological field by providing and utilizing highly informative real-time biofeedback to reliably monitor therapy progress and dynamically adjust therapy session parameters. Specifically, the present invention provides a solution that monitors the reduction of avoidance behavior caused by anxiety and fear invoked by exposure to trauma-triggering situations, and further utilizes it as a feedback to alter therapy parameters. Such a solution allows users to influence the therapy session through their interaction with trauma-associated objects, potentially reducing feelings of helplessness associated with PTSD. Furthermore, it enables therapists to have better control over the progress of the therapy session. Consequently, this solution significantly increases the safety and effectiveness of exposure therapy, leading to improved therapeutic outcomes for individuals with PTSD.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A system for treating a mental condition, comprising:
an input means comprising at least one eye-tracking sensor configured to detect gaze points each representing a data sample indicating where a user is looking at a specific moment in time;
an output means, configured to provide sensory stimuli to the user, said sensory stimuli being characterized by at least one parameter;
at least one non-transitory memory device, wherein modules of instruction code are stored;
and at least one processor associated with said at least one memory device, the input means and the output means, and configured to execute the modules of instruction code, whereupon execution of said modules of instruction code, the at least one processor is configured to:
based on the detected gaze points, continuously calculate a dwell time metric value; and
dynamically alter a value of said at least one parameter, based on the calculated dwell time metric value.

2. The system of claim 1, wherein the dwell time metric value represents a duration of a dwell time period characterized by having the determined gaze points substantially within a specific area of interest (AOI).

3. The system of claim 2, wherein said AOI is characterized by a fixed position in spatial coordinates within the field of view, so that said fixed position remains constant regardless of user’s head movement.

4. The system of claim 2, wherein said at least one processor is further configured to calculate the dwell time metric value by:
defining a set of the gaze points determined withing a sliding time window of a predetermined duration;
determining, within the defined set, one or more sequences of the gaze points characterized by positioning within the AOI;
calculating duration of one or more dwell time sub-periods, each corresponding to a respective one of the determined sequences; and
determining the duration of the dwell time period, based on the calculated duration of the one or more dwell time sub-periods.

5. The system of claim 4, wherein said duration of the dwell time period is represented by at least one of (i) a total duration of all of said one or more dwell time sub-periods; (ii) an average duration of said one or more dwell time sub-periods; and (iii) a standard deviation of the duration of said one or more dwell time sub-periods.

6. The system of claim 4, wherein said at least one processor is further configured to dynamically alter the value of said at least one parameter correlatively to the determined duration of the dwell time period.

7. The system of claim 4, wherein said at least one processor is further configured to dynamically alter the value of said at least one parameter, provided that the determined duration of the dwell time period is below a predefined threshold value.

8. The system of claim 4, wherein said at least one processor is further configured to gradually adjust said predetermined duration of the sliding time window, correlatively to the determined duration of the dwell time period.

9. The system of claim 2, wherein said sensory stimuli comprise visual stimuli.

10. The system of claim 9, wherein said at least one parameter is blurriness.

11. The system of claim 9, wherein said output means comprise a Virtual-Reality (VR) headset configured to display a stereoscopic video content to the user and to track user’s head movement to adjust the stereoscopic video content based thereon.

12. The system of claim 11, wherein said AOI is associated with a virtual object provided within the stereoscopic video content.

13. The system of claim 11, wherein said input means comprise at least one motion controller configured to provide a motion input data element corresponding to the movement thereof, enabling interaction with the stereoscopic video content; and
wherein said stereoscopic video content comprises interactive tasks to be performed using said motion input data element, said interactive tasks requiring the user to retain visual focus withing the AOI in order to successfully complete said interactive tasks.

14. The system of claim 11, wherein said mental condition is Post-Traumatic Stress Disorder (PTSD), and
wherein said AOI is associated with a virtual object provided within the stereoscopic video content, said virtual object being associated with a traumatic experience of the user.

15. A method for treating a mental condition, the method comprising:
detecting, using at least one eye-tracking sensor, a plurality of gaze points each representing a data sample indicating where a user is looking at a specific moment in time;
based on the detected plurality of gaze points, continuously calculating a dwell time metric value; and
providing, using output means, sensory stimuli to the user, said sensory stimuli being characterized by at least one parameter, while dynamically altering a value of said at least one parameter, based on the calculated dwell time metric value.

16. The method of claim 15, wherein the dwell time metric value represents a duration of a dwell time period characterized by having the determined gaze points substantially within a specific area of interest (AOI).

17. The method of claim 16, wherein said AOI is characterized by a fixed position in spatial coordinates within the field of view, so that said fixed position remains constant regardless of user’s head movement.

18. The method of claim 16, wherein said continuously calculating a dwell time metric value comprises:
defining a set of the gaze points determined withing a sliding time window of a predetermined duration;
determining, within the defined set, one or more sequences of the gaze points characterized by positioning within the AOI;
calculating duration of one or more dwell time sub-periods, each corresponding to a respective one of the determined sequences; and
determining the duration of the dwell time period, based on the calculated duration of the one or more dwell time sub-periods.

19. The method of claim 15, wherein said mental condition is Post-Traumatic Stress Disorder (PTSD), and
wherein said AOI is associated with a virtual object provided within a stereoscopic video content, said virtual object being associated with a traumatic experience of the user.

20. The method of claim 15, further comprising:
administering an initial dose of a psychedelic compound to the user,
monitoring, during a predetermined treatment efficiency evaluation period, the calculated dwell time metric value to determine an extent of changes therein;
adjusting the initial dose based on the determined extent of changes; and
administrating the adjusted dose of the psychedelic compound to the user.
